(19) United States
(12) Patent Application Publication
(10) Pub. No.: US 2023/0364284 A1
(43) Pub. Date: Nov. 16, 2023

(54) REUSABLE FOOD OR BEVERAGE CONTAINER SANITIZING SYSTEM

(71) Applicant: Redi Technology, Inc., Bedford, MA (US)

(72) Inventors: Laurence B. Sperry, Newton, MA (US); Brian A. Murch, Needham, MA (US)

(21) Appl. No.: 18/119,399

(22) Filed: Mar. 9, 2023

Related U.S. Application Data
(60) Provisional application No. 63/341,615, filed on May 13, 2022.

(51) Int. Cl. A61L 2/10; A61L 2/24; A61L 2/26

(52) U.S. Cl. CPC A61L 2/10; A61L 2/24; A61L 2/26; A61L 2202/11; A61L 2202/121; A61L 2202/122; A61L 2202/14; A61L 2202/23

(57) ABSTRACT

A reusable food or beverage container sanitizing system and method of operation automates the identification and sanitizing of reusable food or beverage containers. A reusable food or beverage container has an identification device associated therewith. The reusable food or beverage container is associated to a customer order and used to supply a food or beverage to the customer. The system utilizes a UV sanitizing system to prevent the spread of harmful germs on the reusable food or beverage container. The doors of a pass-through UV sanitizing system allow the customer to insert an unsanitized container on the entry (“unsanitized”) side of the system, and the retail employee to remove the sanitized container from the exit (“sanitized”) side of the system without having to tough any other surface (doors, knobs, handles, etc.) than the container itself.

### CROSS-REFERENCE TO RELATED APPLICATIONS

This application claims priority to, and the benefit of, pending U.S. Provisional Application No. 63/341,615, filed May 13, 2022, for all subject matter contained in said application. The disclosure of said application is hereby incorporated by reference in its entirety.

### FIELD OF THE INVENTION

The present invention relates to a reusable food or beverage container sanitizing system that automates the process of identifying and sanitizing reusable food or beverage containers. In particular, the present invention relates to a reusable food or beverage container, an ultraviolet (UV) sanitizing subsystem, and an identification subsystem, which operate in combination with the reusable food or beverage container sanitizing system.

### BACKGROUND

With the rapid expansion and size of the to-go food or beverage industry, for example by global businesses including national and international coffee and fast-food chain restaurants, the disposal of single-use drinking containers, especially containers made with plastics, has become a serious problem. This issue is becoming more than just an environmental or public relations issue for businesses as municipalities have begun to impose taxes on the use of single-use drinking containers. Solutions have been proposed and attempted by manufacturing cups with more environmentally-friendly materials, such as more readily recycled or composted materials. However, the required materials and manufacturing methods have not proven cost-effective as a replacement for existing, commonly used single-use drinking containers. It has also been proven to be difficult to emulate existing desired performance and features using alternate materials.

Systems to provide a means of using reusable drinking containers in place of single-use containers for to-go use have also been proposed and attempted. Such systems are typically comprised of a stock of durable reusable containers which require a deposit from the customer to use for their beverage. When the customer returns the drinking container, the deposit is returned to them. Such systems are typically easy for a customer but increase the labor and required equipment for the retailer to handle and wash the used drinking containers before they can be used again. Such increases have made it a challenge for existing retailers to overcome the cost and infrastructure change of implementing the use of such systems.

Coffee shops, cafes, and other retail establishments that serve beverages often allow customers to bring in their own reusable coffee cups or travel mugs. This trend has a positive impact on reducing the total number of single-use or reusable cups used. Unfortunately, proper cleaning and sanitizing of these reusable cups such that they are safe to be handled and filled by retail employees requires extensive resources. Additionally, the time and effort required to properly clean and sanitize the cups often prevent users from choosing to bring their own reusable containers. Incentives, such as discounts on drinks, are often used to encourage customers to bring their own reusable cups. However, these incentives are often not enough to overcome the perceived inconvenience by customers, such as not being able to order prior to arrival or making use of automatic payments using a mobile application.

### SUMMARY

There is a need for a system for managing the use of multi-use or reusable food or beverage containers that is simple and easy to use for both the customer and the retailer. The present invention provides a system for the use of reusable food or beverage containers, such as for the to-go industry, that closely matches the existing behavior of the customer and retailer such that it can easily be deployed at existing retailers worldwide. An identification subsystem identifies the customer's reusable food or beverage container and the system associates the customer's order to that reusable container. The reusable container is sanitized by the UV sanitizing subsystem such that the reusable container can safely be handled by the retailer employees without concern for the spread of germs. The container is then filled with the food or beverage ordered and returned to the customer.

In accordance with example embodiments of the present invention, a reusable food or beverage sanitizing system is provided. The reusable food or beverage sanitizing system includes a reusable food or beverage container, and identification subsystem, an order information device, and a UV sanitizing subsystem. The identification subsystem includes an identification device associated with the reusable food or beverage container, at least one reader that communicates with the identification device and exchanges container data that characterizes the identification device and associated reusable food or beverage container, and a computing device in communication with a data store storing information relating a food or beverage order to be fulfilled in the reusable food or beverage container. The UV sanitizing subsystem includes a housing sized, dimensioned, and configured to envelop a reusable food or beverage container and at least one UV light source configured to direct UV light at a predetermined intensity level and time duration onto a reusable food or beverage container in such a way as to result in sanitizing of surfaces of the reusable food or beverage container. The UV sanitizing subsystem receives an unsanitized reusable food or beverage container. The UV sanitizing subsystem sanitizes the received reusable food or beverage container prior to the food or beverage order being fulfilled in the reusable food or beverage container. The food or beverage order is fulfilled in the sanitized reusable food or beverage container according to information provided by the order information device in communication with the data store.

In accordance with aspects of the present invention, the at least one reader is disposed to read the identification device prior to placing the reusable food or beverage container into the UV sanitizing subsystem. The identification device can be incorporated on or embedded within the reusable beverage container. An instance of the at least one reader can be disposed to read the identification device of the reusable food or beverage container when placed into the UV sanitizing subsystem. An instance of the at least one reader can be disposed to read the identification device of the reusable food or beverage container prior to returning the filled container to the customer. The identification device can be comprised of one or more of a bar code, a quick response (QR) code, a near-field communication (NFC) chip, or a radio-frequency identification (RFID) tag.

In accordance with aspects of the present invention, the order information device can be a receipt or label printer. In accordance with aspects of the invention, the order information device can be a display screen.

In accordance with aspects of the present invention, an access door can provide access to the UV sanitizing subsystem when the access door is in an open state and block access to the UV sanitizing subsystem when the access door is in a closed state. When the access door is in the closed state, a UV sanitization cycle can be initiated using the UV light source and when the UV sanitization cycle is complete, the access door is moved to an open state. A retrieval door can be located opposite the access door to provide access to the UV sanitizing subsystem when the retrieval door is in an open state and block access to the UV sanitizing subsystem when the retrieval door is in a closed state. The access door can be configured to receive an unsanitized reusable food or beverage container and the retrieval door can be configured to allow retrieval of a sanitized reusable food or beverage container after the UV sanitization cycle. A conveyor can be configured to receive an unsanitized reusable food or beverage container, move the unsanitized reusable food or beverage container in the UV sanitizing subsystem through the access door, and move the sanitized reusable food or beverage container out of the UV sanitizing subsystem through the retrieval door. The conveyor can be comprised of a conveyor belt.

In accordance with aspects of the present invention, a container centering device can be disposed within the UV sanitizing subsystem. The container centering device can be comprised of at least one mechanical arm.

In accordance with aspects of the present invention, the at least one UV light source can be comprised of an array of UV lights. The array of UV light can be disposed on an inner surface of one or more sides of the UV sanitizing subsystem. The at least one UV light source can be disposed on a container profiler that is configured to conform to a shape of the reusable food or beverage container to place the at least one UV light source in proximity to an outer surface of the reusable food or beverage container allowing for effective sanitization at a lower intensity level or time duration. The profiler can be comprised of one or more members having at least one UV light source mounted thereon, the members configured to move toward an outer surface of the reusable food or beverage container to place the UV light in proximity to the outer surface of the reusable food or beverage container. The container profiler can be comprised of a plurality of linear moving members. The container profiler can be comprised of articulating members. The profiler can be comprised of one or more sensors for detecting a shape of the reusable food or beverage container. The container profiler can be configured to rotate around the container to distribute application of UV light.

In accordance with aspects of the present invention, the UV sanitizing subsystem can include a rotating base for rotating the reusable food or beverage container within the UV sanitizing subsystem.

In accordance with aspects of the present invention, the at least one UV light source can be disposed on an arm that extends into an internal volume of the reusable food or beverage container to sanitize an interior surface of the container. The arm can be configured to place the UV light source in proximity to the interior surface of the reusable food or beverage container allowing for effective sanitization at a lower intensity level or time duration. The arm can be configured to rotate within the internal volume of the container to distribute the application of UV light.

In accordance with aspects of the present invention, the housing can rotate around the UV sanitizing subsystem and wherein the access door connected to the housing receives unsanitized reusable food or beverage container in a first position located on a first side of the UV sanitizing subsystem and rotates to a second position located on second side of the UV sanitizing subsystem to provide access to a sanitized reusable food or beverage container.

In accordance with aspects of the present invention, sliding members of the housing can rotate around the UV sanitizing subsystem and wherein the sliding members provide an access door that receives unsanitized reusable food or beverage container in a first position located on a first side of the UV sanitizing subsystem and rotate to a second position located on second side of the UV sanitizing subsystem to provide access to a sanitized reusable food or beverage container.

In accordance with aspects of the present invention, the housing includes a cleansing device.

In accordance with aspects of the present invention, a second UV sanitizing subsystem can be located inside the housing that is sized, dimensioned, and configured to envelop a reusable food or beverage container. A second access door can provide access to the second UV sanitizing subsystem when the second access door is in an open state, and block access to the second UV sanitizing subsystem when the second access door is in a closed state. At least one UV light source can be disposed within the second UV sanitizing subsystem and configured to direct UV light at a predetermined intensity level and time duration onto the reusable food or beverage container positioned inside the second UV sanitizing subsystem in such a way as to result in sanitizing of surfaces of the reusable food or beverage container. When the second access door is in the closed state a UV sanitization cycle can be initiated using the UV light source and when the UV sanitization cycle is complete, the second access door can be moved to the open state. The second UV sanitizing subsystem can be configured to operate in an opposite direction of the UV sanitizing subsystem.

In accordance with aspects of the present invention, the access door can be locked during the sanitization cycle and unlocked after the sanitization cycle. The access door can automatically transition between the closed and open states.

In accordance with example embodiments of the present invention, a method of enabling the use of reusable food or beverage containers using a reusable food or beverage container sanitizing system is provided, a method of enabling the use of reusable food or beverage containers using a reusable food or beverage container sanitizing system includes providing a reusable food or beverage container sanitizing system comprising a reusable food or beverage container, an identification subsystem with at least one reader, an order information device, and a UV sanitizing subsystem. The reusable food or beverage container includes an associated identification device readable by the at least one reader. The system receives a customer order and reads the identification device associated to the customer's reusable food or beverage container such that the system associates the customer order to that reusable food or beverage container. The customer places the reusable food or beverage container into the UV sanitizing subsystem which sanitizing the reusable food or beverage container. The retail employee retrieves the sanitized reusable food or beverage container from the UV sanitizing subsystem and fulfills the customer order in the sanitized reusable food or beverage container according to information provided by the order information device. The customer retrieves the fulfilled order in the reusable food or beverage container from a customer pickup location.

In accordance with aspects of the present invention, the method for associating the reusable food or beverage container identification device with the customer order can occur in a computer executed data store using one or more computing devices to create a data association between the reusable beverage container identification device and the customer order. The one or more computing devices can be located on premise with the system, or remotely in a cloud supported network environment.

In accordance with aspects of the present invention, the method can further include the identification device associated with the reusable food or beverage container that has been previously associated with a customer account and saved in the data store. A customer order can be automatically associated with the identification device of a reusable food or beverage container according to information previously saved in the data store. The identification device can be disposed on or embedded within the reusable food or beverage container. The reader can be disposed within the UV sanitization device such that the identification device of the reusable food or beverage container can be read when the container is placed into the UV sanitizing subsystem and the associated order information is sent to the order information device. The reader can be disposed within the UV sanitization device such that the identification device of the reusable food or beverage container can be read when the container is placed into the UV sanitizing subsystem and an order previously saved in the data store is automatically placed and the associated order information is sent to the order information device.

In accordance with aspects of the present invention, the method can include the reader or other sensor disposed within the UV sanitization device to detect the presence of the reusable food or beverage container and automatically close the access door, run the UV sanitization cycle, and open the retrieval door once the UV sanitization cycle is complete.

In accordance with aspects of the present invention, the method can include a notification to the customer when their order is ready to be retrieved from the customer pickup location. A reader can be used to read the identification device on or embedded within the filled reusable food or beverage container such that the system can notify the customer that their order is ready to be retrieved from the customer pickup location.

### BRIEF DESCRIPTION OF THE FIGURES

These and other characteristics of the present invention will be more fully understood by reference to the following detailed description in conjunction with the attached drawings, in which:

FIG. 1 is a diagrammatic illustration of a reusable food or beverage container sanitizing system, according to embodiments of the present invention;

FIG. 2 is an isometric view of a housing of the reusable food or beverage container sanitizing system containing a UV sanitizing subsystem in accordance with embodiments of the present invention;

FIGS. 3A-3C are isometric views of a housing of the reusable food or beverage container sanitizing system containing a UV sanitizing subsystem in accordance with embodiments of the present invention;

FIGS. 4A-4C are isometric views of a housing of the reusable food or beverage container sanitizing system containing a UV sanitizing subsystem with conveyance in accordance with embodiments of the present invention;

FIG. 5 is an isometric cut-away view of a housing of the reusable food or beverage container sanitizing system containing a UV sanitizing subsystem with conveyance and centering arms in accordance with embodiments of the present invention;

FIGS. 6A-6C are isometric cut-away views of a housing of the reusable food or beverage container sanitizing system containing a UV sanitizing subsystem with a profiler in accordance with embodiments of the present invention;

FIGS. 7A-7B are isometric cut-away views of a housing of the reusable food or beverage container sanitizing system containing a UV sanitizing subsystem with a profiler having multiple linear moving members in accordance with embodiments of the present invention;

FIG. 8 is an isometric view of a revolving housing of the reusable food or beverage container sanitizing system containing a UV sanitizing subsystem with a profiler having multiple articulating members in accordance with embodiments of the present invention;

FIG. 9 is an isometric cut-away view of a housing of the reusable food or beverage container sanitizing system containing a UV sanitizing subsystem with a rotating base and a UV light source disposed on an arm that extends into the interior volume of the container in accordance with embodiments of the present invention;

FIGS. 10A-10C are isometric views of a housing of the reusable food or beverage container sanitizing system containing two UV sanitizing subsystem in accordance with embodiments of the present invention;

FIG. 11 is an isometric view of an example reusable food or beverage container for use in the system;

FIG. 12 is a flow diagram illustrating operation of a reusable food or beverage container sanitizing system;

FIG. 13 is a flow diagram illustrating operation and automated processes of a reusable food or beverage container sanitizing system; and

FIG. 14 is a diagrammatic illustration of a computing device and related hardware for use in implementation of the present invention.

### DETAILED DESCRIPTION

An illustrative embodiment of the present invention relates to a reusable food or beverage container sanitizing system that automates the process of identifying and sanitizing reusable food or beverage containers. The system provides a simple, efficient, and easy to use method for the implementation of the reusable food or beverage containers to replace the current prevalent use of wasteful and environmentally harmful single-use cups and take-out food containers that require recycling or disposal.

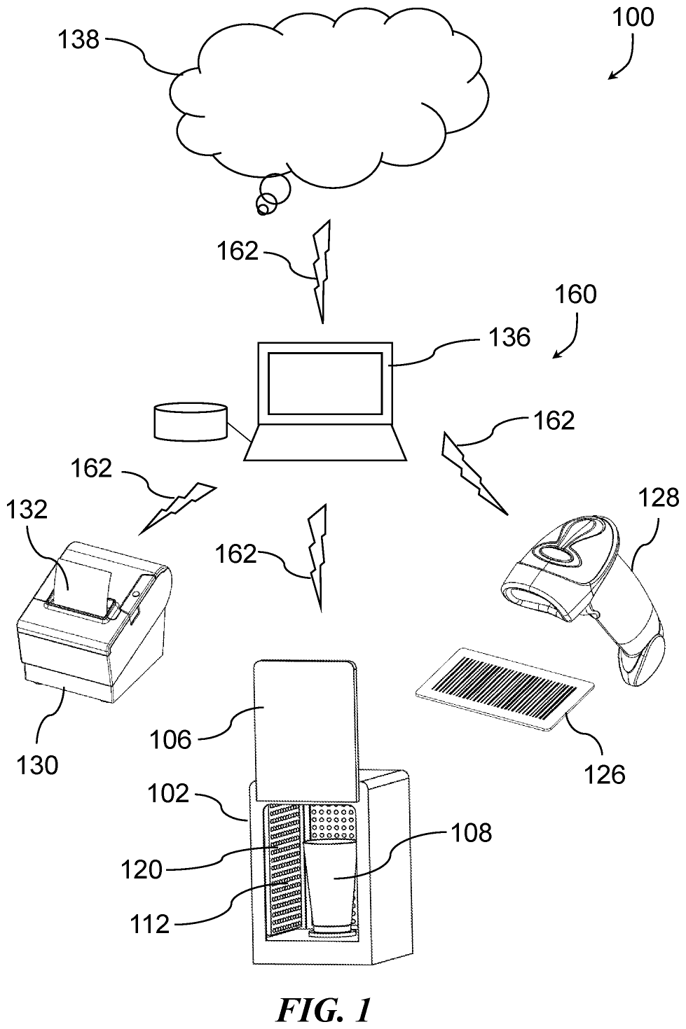

FIGS. 1 through 14, wherein like parts are designated by like reference numerals throughout, illustrate an example embodiment or embodiments of a reusable food or beverage container sanitizing system, according to the present invention. Although the present invention will be described with reference to the example embodiment or embodiments illustrated in the figures, it should be understood that many alternative forms can embody the present invention. One of skill in the art will additionally appreciate different ways to alter the parameters of the embodiment(s) disclosed, such as the size, shape, or type of elements or materials, in a manner still in keeping with the spirit and scope of the present invention.

FIG. 1 shows a diagrammatic illustration of a reusable food or beverage container sanitizing system 100 in accordance with embodiments of the present invention. The example reusable food or beverage container sanitizing system 100 depicted in the figures uses a reusable beverage container 108 for the description and figures merely for purposes of teaching how the invention can be specifically implemented. However, those of skill in the art will readily appreciate that other reusable food or beverage containers can be implemented in the system 100 described herein with modification to some of the components for the specific size, shape, and configuration of the containers, such that the present invention is intended to be universally applicable and operable for a variety of different food or beverage container configurations and is not limited to the specific reusable beverage container shown and described. It should also be appreciated that for purposes of efficiency, the references herein to a reusable beverage container and corresponding system are fully applicable to all possible food or beverage containers and corresponding systems. For the avoidance of doubt, references to the reusable beverage container and corresponding system herein are transferable to other food or beverage containers, such as but not limited to, pizza boxes, Asian food takeout containers, donut boxes, pastry boxes, to-go restaurant food containers, coffee cups, iced coffee cups, hot chocolate cups, water bottles, other disposable beverage containers, and the like, which are considered to all fall within the scope of the present inventive system.

Continuing with the example system 100, a housing 102 can contain a number of subsystems including an access door 106 for receiving a reusable beverage container 108 and a UV sanitizing subsystem 112 disposed therein. The UV sanitizing subsystem 112 contains at least one UV light source 120, which uses UV-C(or other wavelength) to sanitize items during a UV sanitization cycle. At least one reader 128 that communicates with an identification device 126 associated to the reusable beverage container 108, and exchanges data that characterizes the identification device 126 and associated reusable beverage container 108. A computing device 136 in communication with a data store 138, such as a database or other form of data storage and organization, is a portion of an identification subsystem 160 that also includes the identification device 126, the at least one reader 128, and a communication network 162 that enables communication between the components of the identification subsystem 160 and sharing of data. The communication network 162 can be a local area network, a wide area network, wired, wireless, cloud-based, or any other network or data sharing construct understood by those of skill in the art. The computing device 136 can be on premise at the same location as, e.g., the housing 102, or remote in a network, cloud supported, wireless, or wired. Those of skill in the art will additionally appreciate that the location of each subsystem as depicted and described herein can vary such that subsystems may be combined, integrate, and/or distal, separated, in different combinations and still perform the stated functionality and operation described herein, all of which combinations are anticipated to fall within the scope of the present invention. The specific hardware and device arrangements described herein are merely representative of one example implementation of the inventive system and method and are therefore not intended to be limiting.

Continuing with FIG. 1 illustrating the housing 102 that can contain a number of subsystems. As one of skill in the art would appreciate, various configurations of subsystems can be implemented keeping with the spirit and scope of the present invention, including having those subsystems contained inside the housing 102 or separated therefrom. In accordance with one exemplary illustrative embodiment, the reusable food or beverage container sanitizing system 100 has an access door 106 or the like of any operable configuration. The access door 106 is sized, dimensioned, and configured to receive a reusable beverage container 108. In the example illustration, the reusable beverage container 108 is a conventional reusable coffee beverage cup and as such has dimensions that can vary but are generally about 2.25 inches to 2.5 inches for the small diameter, about 3 inches to 3.75 inches for the large diameter, and between about 3.25 inches to about 8 inches of height, for containers that range from, e.g., 6 oz to 22 oz of volume. Those of skill in the art will appreciate these dimensions are approximate and are intended to describe the approximate size and shape of conventional coffee cups, or the like, which conventionally fit in conventional automobile cupholders, and can be found in numerous coffee retail stores. As such, the access door 106 and UV sanitizing subsystem 112 must be at least slightly larger than the maximum dimensions of the aforementioned cups (e.g., for the above example dimensions, the access door 106 has an opening of about 5 inches wide by 10 inches high to accommodate the largest cup measurements). The access door 106 can automatically slide open, or flap shut like a one-way valve, or have another configuration readily apparent to those of skill in the art. In another embodiment, the access door 106 and UV sanitizing subsystem 112 are sized to accept a reusable beverage container 108 and attached corresponding lid. In another embodiment, the access door 106 and UV sanitizing subsystem 112 are sized to accept both a reusable beverage container 108 and a separated corresponding lid. In yet another embodiment, the access door 106 and UV sanitizing subsystem 112 are sized to accept more than one reusable beverage container 108 and/or lid simultaneously. Furthermore, those of skill in the art will appreciate the dimensional attributes are provided for enablement purposes only as it relates to a system for use with a coffee beverage container or the like and can be altered or varied to accommodate different containers of different dimensions for different purposes, and are therefore not limiting of the system-wide invention.

After a customer places an order for a beverage to be fulfilled in the customer's reusable beverage container 108, such as in person at the point-of-sale register or a self-serve kiosk, or remotely orders via mobile application, online ordering, and the like, the identification device 126 to be associated to the desired reusable beverage container 108 is read by a reader 128 of the identification subsystem 160 to associate the customer's order to the reusable beverage container 108 in which that order is to be fulfilled. In an embodiment of the invention, the identification device 126 is an optical identifier, such as a barcode, quick response (QR) code, optical symbol or character recognition, and the like. In another embodiment, the identification device 126 is a transmission protocol identifier, such as a near-field communication (NFC) chip, radio frequency identification (RFID) tag, and the like. In an embodiment, the optical or transmission protocol identifier that is the identification device 126 is on or embedded in a possession of the customer, such as a loyalty card, key fob, mobile phone, and the like. In another embodiment, the optical or transmission protocol identifier that is the identification device 126 is on or embedded within the reusable beverage container 108 itself. In yet another embodiment, the identification device 126 is biometric, such as a fingerprint or facial recognition of the customer. In still a further embodiment, the identification device 126 is manually entered customer information, such as manually typing in the customer's name, loyalty number, credit card information, and the like, into the point-of-sale register, self-serve kiosk, or mobile or online ordering device.

In an embodiment of the invention, the reader 128, such as a scanner, camera, NFC reader, and the like, is located at the point-of-sale register or kiosk. In another embodiment, the reader 128 is a feature of the customer's mobile phone, such as the camera, NFC reader, and the like. In a further embodiment, the reader 128 is integral to the housing 102. Depending on the desired configuration, the reader 128 can be in communication with a processor and/or local or remote computing system via wired or wireless network connectivity, such as the computing device 136 of the identification subsystem 160, and the like, as would be readily appreciated by those of skill in the art.

In an embodiment of the invention, the identification subsystem 160 can include a camera to capture a photograph of the reusable beverage container 108 to be saved with other identification information of the reusable beverage container 108 in the data store 138.

Once an association has been created in the system 100 between a customer and the identification device 126 of a reusable beverage container 108, that link can be saved in the data store 138 such that a new association does not need to be established for subsequent orders using that particular reusable beverage container 108. As such, when an order is placed by a customer, which can be identified by a name, customer account, member number, phone number, loyalty card, signed into an application for ordering on a mobile device, fingerprint reader, facial recognition, and the like, the established association between the customer's order and their reusable beverage container 108 is automatically created without having to newly create the association between the identification device 126 and the reusable beverage container 108 as previously described. If the customer has more than one reusable beverage container 108 associated with them, they can select which associated reusable beverage container 108, will be used for that order, such as by selecting a photograph of the desired reusable beverage container 108 at the point-of-sale register, kiosk, on the mobile application or online order, or at the UV sanitizing subsystem 112. In a similar fashion, if the customer orders multiple beverages to be fulfilled in more than one reusable beverage container 108, they can select which order is to be fulfilled in which known reusable beverage container 108 as previously described. Additionally, if only a portion of an order for multiple beverages is to be fulfilled in a known reusable beverage container 108, the customer can choose to create a new association for an unknown reusable beverage container 108 for other portions of the order and/or have other portions of the order filled in traditional single use cups or other options available by the retailer.

As many customers place the same order every time, the customer can choose to have that particular order associated with one or more reusable beverage container 108. This association can allow the customer to skip the ordering process completely as the saved order, as well as required payment if desired, can be placed automatically when the identification device 126 of the reusable beverage container 108 is identified by a reader 128 of the identification subsystem 160 of the system 100. Additional incentives for customers using the system 100 can be implemented, such as discounts, loyalty rewards, order prioritization, and the like, to encourage the increased use of reusable beverage containers 108.

Once a reader 128 of the identification subsystem 160 has identified the identification device 126 of the reusable beverage container 108, the information for the corresponding order to be fulfilled in that particular reusable beverage container 108 can be output by the order information device 130. The order information device 130 provides information to allow the retail employee to properly fulfill the order for a particular reusable beverage container 108, such as the details of the order itself, description, photograph, or other identifying information of the reusable beverage container 108, customer's name, and the like, as would be appreciated by one of skill in the art. In an embodiment shown in FIG. 1, the order information device 130 is a receipt or label printer configured to print order details on a receipt or label 132 that is to accompany the reusable beverage container 108 after it has been removed from the UV sanitizing subsystem 112. In another embodiment, the receipt or label printer is integral to the housing 102. In another embodiment, at least a portion of the receipt or label 132 is removably self-adhesive such that it may temporarily adhere to the reusable beverage container 108. In a further embodiment, the order information device 130 is a display integral to the housing 102. In another embodiment, the order information device 130 is a remote display or displays at other locations within the retail location. In another embodiment, the order information device 130 temporarily labels the reusable beverage container 108, such as printing on the outer surface of the reusable beverage container 108 using washable ink and the like. In all embodiments, the order information device 130 can be in communication with a processor and/or local or remote computing system via wired or wireless network connectivity, such as the computing device 136 of the system 100, or the like, as would be readily appreciated by those of skill in the art. It should also be appreciated by one of skill in the art that more than one type of order information device 130 may be used simultaneously in keeping with the spirit and scope of the invention.

The reader 128 as shown in FIG. 1 is remotely located outside of the housing 102 and in communication with the system 100 via the communication network 162. In another embodiment, the identification subsystem 160 may contain one or more reader 128. The one or more reader 128 can be integral to the housing 102 or remotely located as previously described. The one or more reader 128 can be configured and located to identify an identification device 126 on or embedded within a reusable beverage container 108 prior to entering the UV sanitizing subsystem 112, when the reusable beverage container 108 is placed into the UV sanitizing subsystem 112, when the reusable beverage container 108 is removed from the UV sanitizing subsystem 112, or any combination of such. In addition to identifying the identification device 126 of the reusable beverage container 108 to have the order information device 130 output details of the corresponding order, the identification subsystem 160 can be used to trigger other automated processes of the system 100. In an embodiment of the system, identifying the identification device 126 of the reusable beverage container 108 prior to entering the UV sanitizing subsystem 112 can unlock or automatically open the access door 106 to the UV sanitizing subsystem 112 or otherwise allow the identified reusable beverage container 108 to be entered into the UV sanitizing subsystem 112. In another embodiment of the system 100, identifying when the reusable beverage container 108 has been placed into the UV sanitizing subsystem 112 can trigger the start of the UV sanitization cycle, such as closing the access door 106, turning on the one or more UV light source 120 for a determined amount of time, and opening the access door 106 when complete. In yet another embodiment, identifying when the sanitized reusable beverage container 108 is removed from the UV sanitizing subsystem 112 can trigger the system to return to the state such that the system is ready to accept the next unsanitized reusable beverage container 108. It should be recognized by one of skill in the art that other means to identify the presence or removal of a reusable beverage container 108, such as proximity sensors, ultrasonics sensors, vision systems, and the like, may be used to trigger other automated processes of the system as previously described.

After the customer has placed their reusable beverage container 108 into the UV sanitizing subsystem 112 and the UV sanitization cycle has been completed, the sanitized reusable beverage container 108 is removed from the UV sanitizing subsystem 112 by the retail employee. In an embodiment of the invention, the retail employee can use an additional cleansing device, such as a countertop glass rinser, to clean residue from the interior surfaces of the reusable beverage container 108. In another embodiment, such an additional cleansing device can be used by the customer prior to placing the reusable beverage container 108 into the UV sanitizing subsystem 112. In yet another embodiment, the additional cleansing device is within the housing 102 such that the UV sanitization cycle and additional cleansing take place within the housing either concurrently or in succession.

After retrieving the sanitized reusable beverage container 108, the retail employee fulfills the order into that reusable beverage container 108 according to information provided by the order information device 130. Once the order is fulfilled, the retail employee returns the filled reusable beverage container 108 to the customer. In an embodiment of the invention, the retail employee places the filled reusable beverage container 108 into a UV sanitizing subsystem 112 of the system 100 to be sanitized and subsequently retrieved by the customer as to prevent the spread of germs from the retail employee to the customer. In a further embodiment, a reader 128 integral to or in close proximity to the housing 102 identifies the identification device 126 on or embedded within the reusable beverage container 108 placed into the UV sanitizing subsystem 112 such that the system 100 can notify the customer that their order is ready to be retrieved. Such notification can be an audio alert (e.g. customer's name called out via a speaker system), a visual alert (e.g. customer's name displayed on a video screen), a text message, phone call, mobile application notification, and the like, as would be appreciated by one of skill in the art. In another embodiment, the filled reusable beverage container 108 is not placed into a UV sanitizing subsystem and is instead placed directly at a customer pick-up location, such as a pick-up counter. In another embodiment, a reader 128 of the system detects the identification device 126 on or embedded within the reusable beverage container 108 either immediately prior to or when the reusable beverage container 108 is placed at the customer pick-up location such that the system can notify the customer that their order is ready to be retrieved as previously described. In a further embodiment, the pickup location is a specific UV sanitizing subsystem or locker, cubby, shelf, or the like. In an additional embodiment, a reader 128 is used to verify the customer, such as by NFC of the customer's phone, scanning of a barcode in a mobile application or on a loyalty card, fingerprint reader, facial recognition, or other identification means as would be appreciated by one of skill in the art, to grant access to the pickup location, such as unlocking or opening an access door. In another embodiment, other methods are used to trigger the system 100 to notify the customer that their order is ready to be retrieved, such as reading information on a label or receipt 132 that was produced by the order information device 130, or otherwise manually updating the status of the order within the system 100 to indicate the order has been fulfilled. In yet another embodiment, the system 100 is not used to alert the customer that their order is ready to be retrieved and other manual methods, such as leaving the label or receipt 132 adhered to or in close proximity to the filled reusable beverage container 108, or the retail employee calling out the customer's name or handing the filled reusable beverage container 108 directly to the customer, are used as would be appreciated by one of skill in the art.

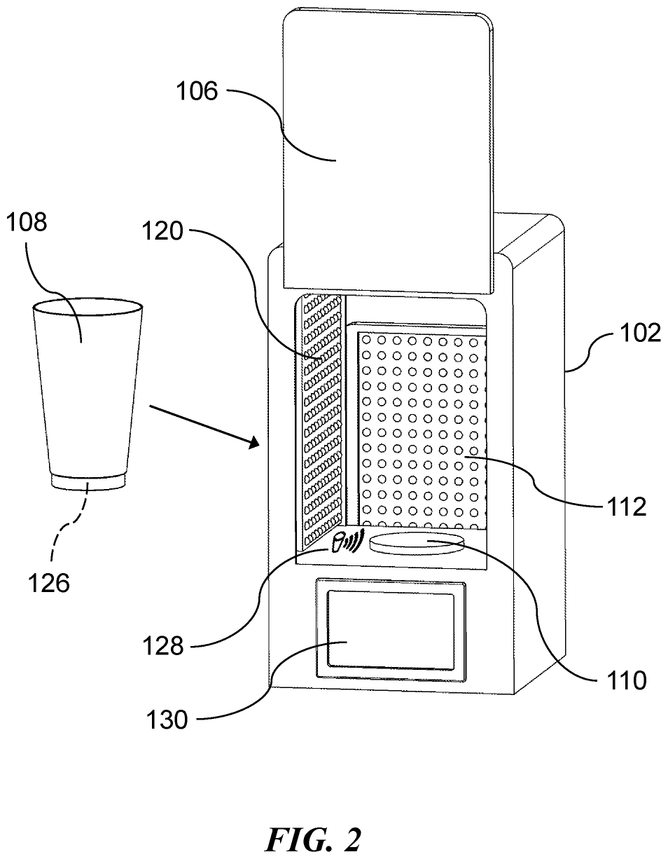

FIG. 2 illustrates an embodiment of the UV sanitizing subsystem 112 with a reader 128 integral to the housing 102 to read the identification device 126 on or embedded within the reusable beverage container 108 placed into the housing 102 such that the identification subsystem 160 (refer to FIG. 1), in communication with the data store 138 of the system 100 (refer to FIG. 1), can create an association between that reusable beverage container 108 and the order to be fulfilled as previously described. In an embodiment of the invention, the reader 128 can be used to automatically place an order and/or charge for payment based on information stored in the data store 138 (refer to FIG. 1) for the reusable beverage container 108 detected as previously described. In another embodiment, detecting the presence of a reusable beverage container 108 with the reader 128 can trigger automatic events or processes of the system, such as opening/closing the access door 106 or starting the UV sanitization cycle as previously described.

The housing 102 as shown in FIG. 2 contains an integral visual display, such as an LCD display, LED display, and the like, as the at least one component of the order information device 130. Such display can provide information to allow the retail employee to properly fulfill the order for the particular reusable beverage container 108, such as the details of the order itself, description, photograph, or other identifying information of the reusable beverage container 108, customer's name, and the like, as previously described.

In embodiments of the invention, the reusable beverage container 108 can be rotated while within the UV sanitizing subsystem 112 to ensure all surfaces are evenly sanitized. In an embodiment as shown in FIG. 2, the UV sanitizing subsystem 112 can contain a rotating base 110. The reusable beverage container 108 is placed on the rotating base 110 such that the rotation of the rotating base 110 will ensure even exposure of the surfaces of the reusable beverage container 108 to the at least one UV light source 120. In another embodiment, the at least one UV light source 120 is rotated around the reusable beverage container 108 during the sanitizing process. In yet another embodiment, both the reusable beverage container 108 and the at least one UV light source 120 are rotated during the sanitization cycle.

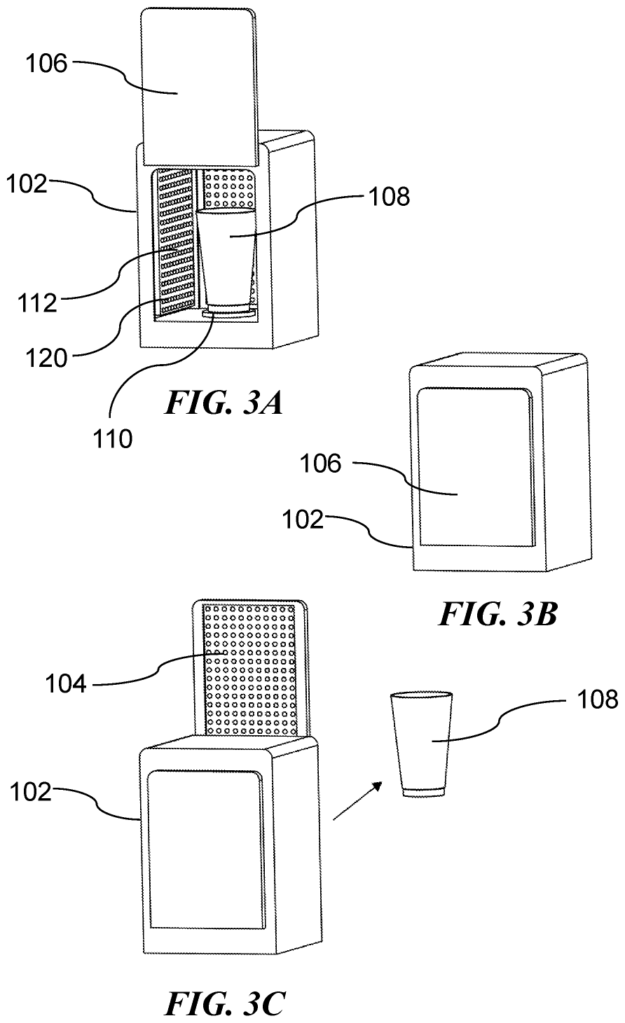

FIGS. 3A-3C illustrates an embodiment of the UV sanitizing subsystem 112 of a reusable beverage container sanitizing system 100 (refer to FIG. 1) that is comprised of a housing 102 that has access to the UV sanitizing subsystem 112 from two sides; the entry (“unsanitized”) having an access door 106 and exit (“sanitized”) side having a retrieval door 104. In FIG. 3A, the customer places their reusable beverage container 108 into the UV sanitizing subsystem 112 from the “unsanitized” side via the access door 106 in an open position. In FIG. 3B the access door 106 is moved to a closed state and the reusable beverage container 108 is sanitized within the enclosed UV sanitizing subsystem 112 during a UV sanitization cycle using at least one UV light source 120. In this embodiment, the at least one UV light source 120 is an array of UV lights disposed on one or more interior surfaces of the UV sanitizing subsystem 112. In FIG. 3C, after the UV sanitization cycle is complete, the retrieval door 104 is moved from a closed state to an open state allowing the retail employee to remove the sanitized reusable beverage container 108 on the “sanitized” side. In some embodiments of the invention, the reusable beverage container 108 or UV light source 120 is rotated to evenly distribute UV light to the surface of the reusable beverage container 108 to be sanitized. In the embodiment as shown in FIGS. 3A-3C, a rotating base 110 is provided to rotate the reusable beverage container 108 within the UV sanitizing subsystem 112 providing more thorough exposure to the array of at least one UV light source 120 on the inner surface of the UV sanitizing subsystem 112 as previously described.

The retrieval door 104 can be sized, configured, and controlled similarly to the access door 106 as previously described. In some embodiments, the access door 106, as well as the retrieval door 104, to the UV sanitizing subsystem 112 can be automated to open and close automatically to eliminate the need to touch anything but the reusable beverage container 108 itself when using the system 100 (refer to FIG. 1). In some embodiments, the automatic opening and closing of the door(s) makes it possible to have a single access door 106, such as on the top of the housing 102, for both entry and exit, without the risk of spreading germs. In some such embodiments, an identification device 126 of the reusable beverage container 108 is read by a reader 128 of the identification subsystem 160 (refer to FIG. 1) and is used to unlock the access door 106 or retrieval door 104, automatically open the door 106, 104, or otherwise be used to initiate the UV sanitization cycle of the UV sanitizing subsystem 112. In still other embodiments, the opening or closing of the doors 106, 104 or the initiation of a UV sanitization cycle is actuated by a switch, button, motion sensor, or optical sensor. Whether manually or automatically closed, the access door 106, as well as the retrieval door 104, can lock during the UV sanitization cycle as a safety precaution to prevent inadvertent UV exposure to a user. Conversely, there can be an automatic shut-off of the one or more UV light source 120 if the access door 106 or retrieval door 104 are opened before a UV sanitization cycle is complete.

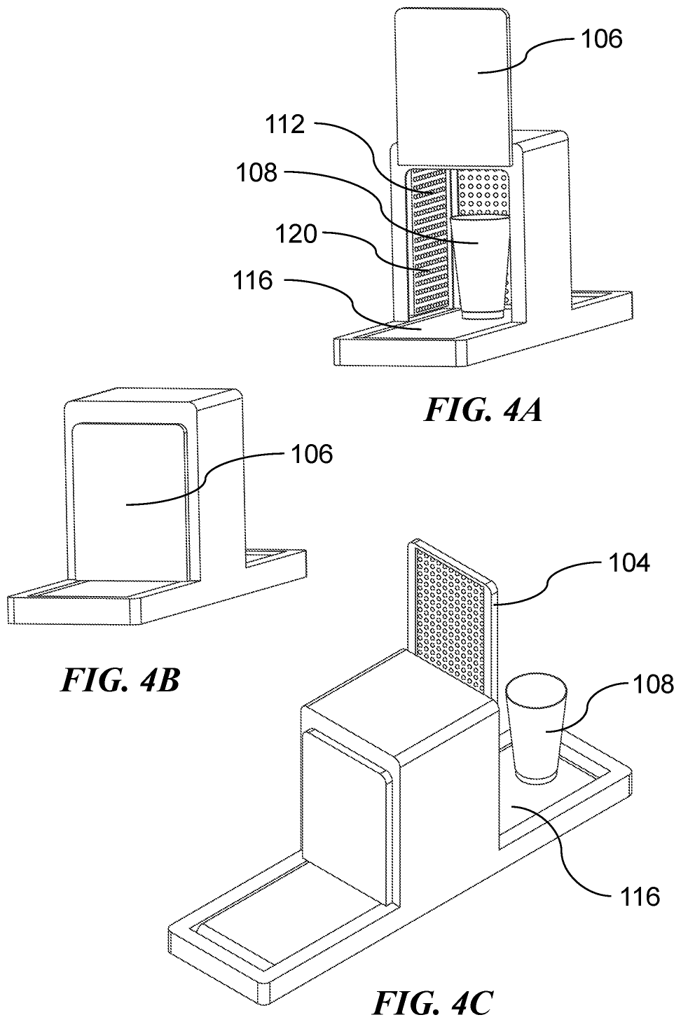

FIGS. 4A-4C depict a UV sanitizing subsystem 112 similar to FIGS. 3A-3C with the addition of a conveyor 116 to move the unsanitized reusable beverage container 108 into the UV sanitizing subsystem 112 on the “unsanitized” side and then move the sanitized reusable beverage container 108 out of the UV sanitizing subsystem 112 on the “sanitized” side. This simplifies the loading and unloading of the UV sanitizing subsystem 112, as well as adds the ability to queue additional reusable beverage containers 108 to be sanitized. In FIG. 4A, the customer places their reusable beverage container 108 onto the conveyor 116, in this case, a conveyor belt. The conveyor 116 then moves the reusable beverage container 108 into the UV sanitizing subsystem 112 from the “unsanitized” side via the access door 106 in an open position. In FIG. 4B the access door 106 is moved to a closed state and the reusable beverage container 108 is sanitized within the enclosed UV sanitizing subsystem 112 during a UV sanitization cycle using at least one UV light source 120. In FIG. 4C, after the UV sanitization cycle is complete, the retrieval door 104 is moved from a closed state to an open state and the conveyor 116 moves the now sanitized reusable beverage container 108 out of the UV sanitizing subsystem 112 through the retrieval door 104 allowing the retail employee to remove the sanitized reusable beverage container 108 from the conveyor 116 on the “sanitized” side.

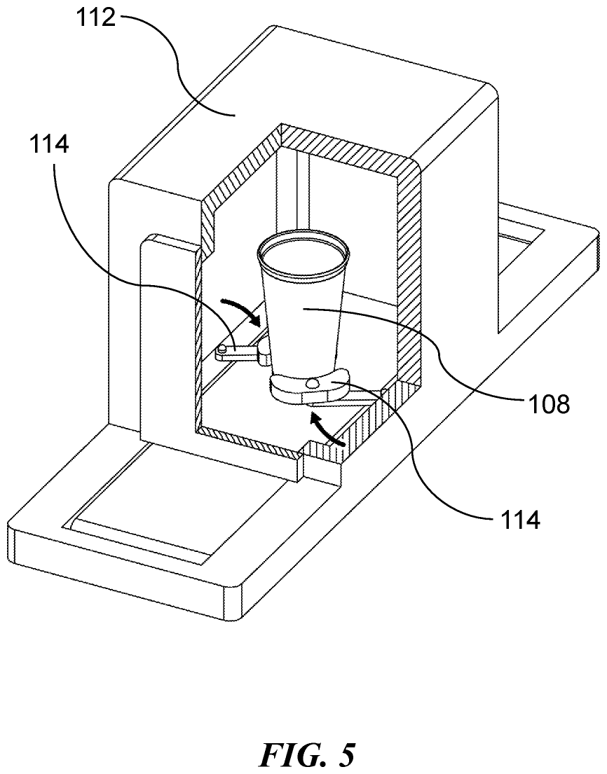

In some embodiments, container centering devices, such as the pivoting centering mechanical arms 114, shown in FIG. 5, are utilized to position the reusable beverage container 108 in the center of the UV sanitizing subsystem 112 to ensure the even and expected distribution and intensity of UV light on the surfaces.

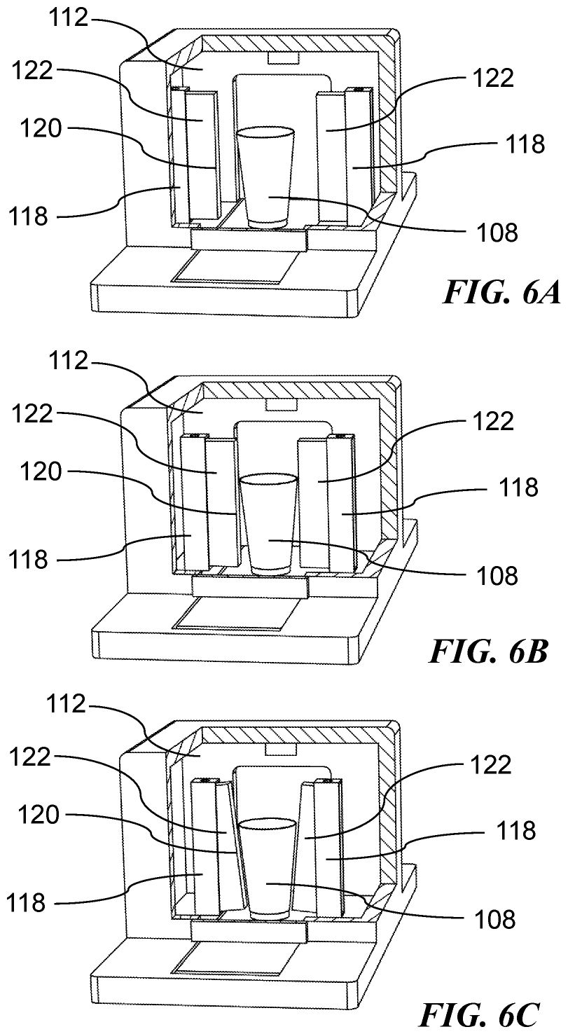

In one embodiment of the UV sanitizing subsystem 112 of the current invention shown in FIGS. 6A-6C, the at least one UV light source 120 is disposed on at least one or more articulating members 122 of a container profiler 118 that is able to move the members 122 including the at least one UV light source 120 close to the outer surface of the reusable beverage container 108 and, if required, adjust or otherwise conform to approximately match the tapered shape of commonly used reusable beverage containers 108. In FIG. 6A, the profiler 118 is set at a starting position before being positioned to conform to the reusable beverage container 108. In the embodiment of FIG. 6B, the profiler 118 has been adjusted to linearly move the members 122 in proximity to the outer surface of the reusable beverage container 108. In certain embodiments, such as seen in FIG. 6C, the members 122 are further capable of moving angularly or otherwise pivoting or tilting to better conform to the profile of the reusable beverage container 108.

It should be noted that any of the embodiments described herein can be alternatively embodied in fixed form. Specifically, and for example, looking at the embodiment depicted in FIG. 6C, this embodiment shows the members 122 angularly positioned to match the angle and profile of the reusable beverage container 108. In the instance where the reusable beverage container 108 will be of a substantially similar shape in every instance of use, the one or more members 122 can be permanently affixed to a shape and/or angle that matches the shape and profile of the reusable beverage container 108 and resemble the UV sanitizing subsystem 112 as shown in this figure. In such an embodiment, the one or more members 122 move around the reusable beverage container 108 in a fixed position, or the reusable beverage container 108 can be rotated if the members 122 are fixed, resulting in the full application of UV light to the reusable beverage container 108 to achieve the stated desired result. This alternative embodiment of fixed members 122 is anticipated herein for all operable embodiments, and all figures provided herein can be alternatively interpreted to show a fixed profile embodiment.

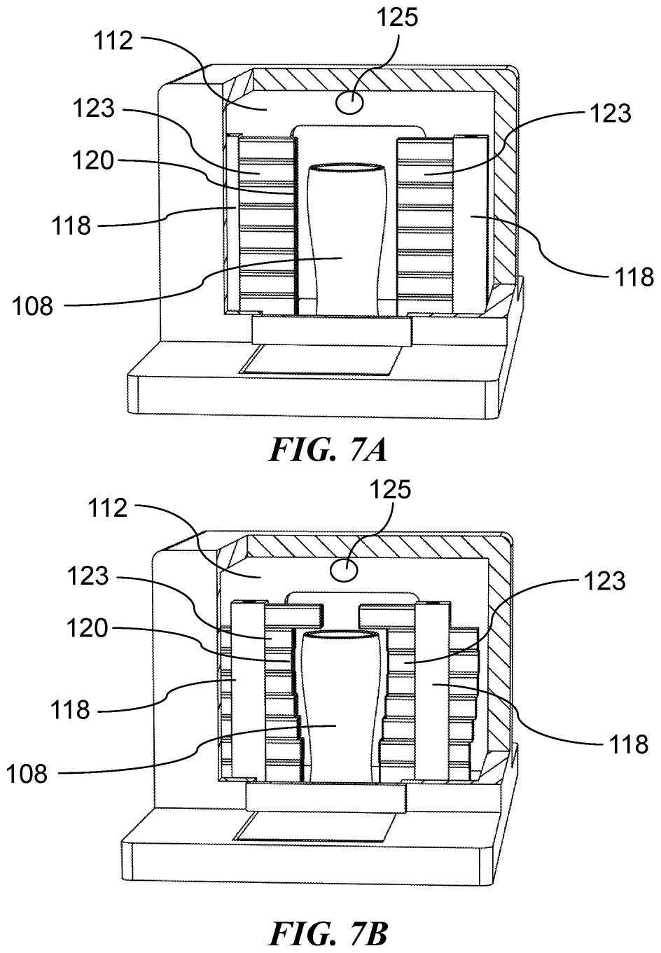

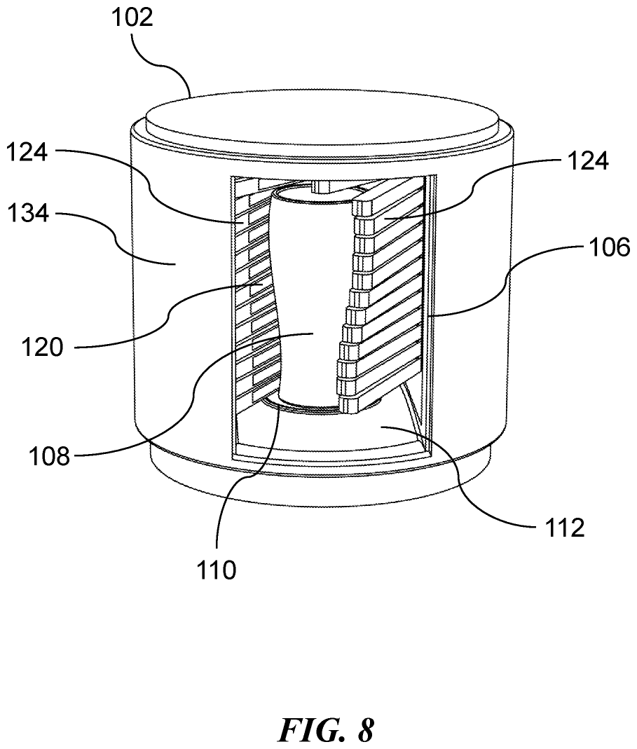

In accordance with example embodiments, the one or more UV light source 120 of the profiler 118 are mounted to multiple movable profiling members 122 which can more accurately adjust to a variety of container profiles, such as the linear motion profiling members 123 shown in FIGS. 7A-7B and the pivoting profiling members 124 shown in FIG. 8. The positioning of the profiling members 122, 123, 124 of the profiler 118 close to the surface of the food or beverage container 108 can be achieved using one or more sensors, such as a vision subsystem 125 shown in FIG. 7A-7B, proximity sensors, or stall sensors incorporated into the profiler 118 used to detect when the profiling members 122, 123, 124 contact the surface of the reusable beverage container 108, then back off slightly. In all embodiments, either the reusable beverage container 108 or one or more UV light source 120 can be rotated, for example with a rotating base 110 as shown in FIG. 8, to ensure all surfaces are evenly and effectively sanitized without requiring additional time or an excessive number of UV light sources 120.

In accordance with example embodiments, such as seen in FIG. 8, the housing 102 or one or more sliding members 134 of the housing 102 providing the access door 106 can be rotated or revolved around the UV sanitizing subsystem 112 to move the access door 106 between an open state providing access to the UV sanitizing subsystem 112 or a closed state blocking access to the UV sanitizing subsystem 112. In embodiments of the invention, the rotating housing 102 or one or more sliding members 134 of the housing can rotate between states of providing access to place a reusable beverage container 108 into the UV sanitizing subsystem 112 from the “unsanitized” side, a closed state to prevent any access during the UV sanitization cycle, and a state providing access to retrieve the reusable beverage container 108 from the “sanitized” side as previously described.

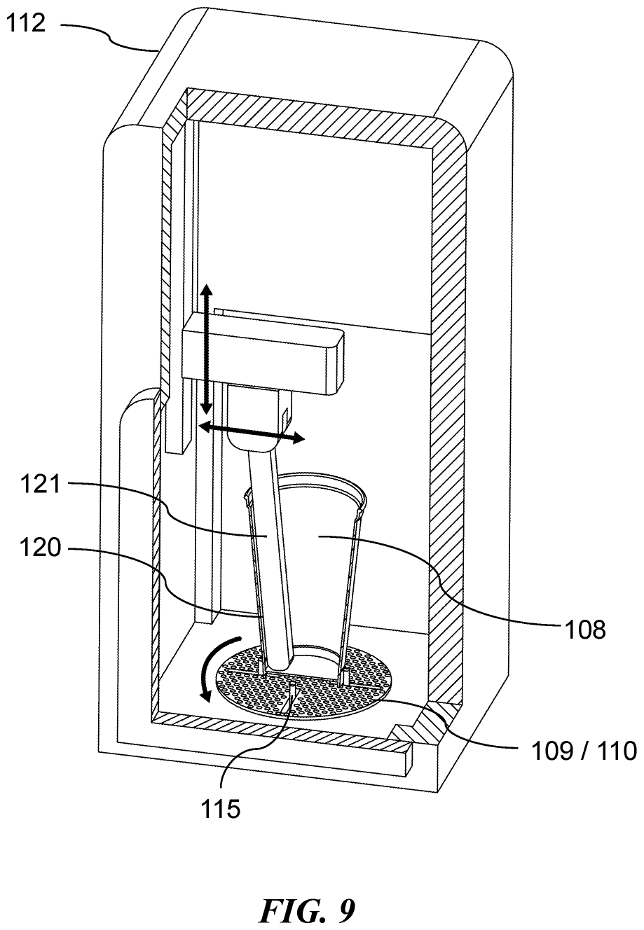

Since the outer surface of the reusable beverage container 108 is what is handled by customers and retail employees, thus the surface most likely to transfer germs, it may be acceptable to only sanitize that surface. Alternatively, it may be desirable to sanitize all surfaces. An illustrative embodiment of the UV sanitizing subsystem 112 as shown in FIG. 9 uses a profiling arm 121 containing an array of at least one UV light source 120, which is lowered into the internal volume of the reusable beverage container 108 and adjusted to position the array of at least one UV light source 120 in close proximity to the interior surface. A centering device, such as the centering fingers 115 on the rotating base 110 of FIG. 9, or pivoting centering mechanical arms 114 as previously shown in FIG. 5, or any similar device, is used to properly position the reusable beverage container 108 such that when rotated on the rotating base 110, the profiling arm 121 maintains a position in close proximity to the surface to ensure proper and effective sanitizing. Positioning of the profiling arm 121 can be performed with the use of one or more sensors, such as a vision subsystem 125 (refer to FIG. 7), proximity sensors, or stall sensors incorporated into the profiling arm 121. The array of at least one UV light source 120 of the interior profiling arm 121 includes UV lights positioned to emit light down onto the interior bottom surface of the reusable beverage container 108 such that all interior surfaces are sanitized. To sanitize the bottom exterior of the reusable beverage container 108, a UV light or UV light array (not shown) may be placed below a perforated base 109, in this case, the perforated rotating base 110 as shown in FIG. 9. The positioning of at least one UV light source 120 below a perforated base 109 and/or movement of the perforated base 109 allows the emitted light to reach all areas of the bottom surface to ensure proper and effective sanitization. It should be noted that any combination of the presented concepts may be combined to achieve a desired level of sanitization of the reusable beverage container 108.

In certain embodiments where a standardized reusable beverage container 108 is used with the UV sanitizing subsystem 112, the profiler 118 (including members 122, 123, 124), profiling arm 121, or the UV sanitizing subsystem 112 itself (refer to FIGS. 6A-8) can have a fixed shape or configuration that conforms to the dimensions of the standardized reusable beverage container 108, such that the one or more UV light source 120 are in close proximity to the surface(s) of the standardized reusable beverage container 108. This would simplify the UV sanitizing subsystem 112 as the profiler 118 would not require one or more sensors such as a vision subsystem 125, proximity sensors, or stall sensors incorporated into the profiler 118 used to detect when the profiling arms contact the surface, then back off slightly.

In still other embodiments, the data store 138 (refer to FIG. 1) can contain information about the shape of the reusable beverage container 108 such that when a reader 128 of the identification subsystem 160 (refer to FIG. 1) reads the identification device 126 of the reusable beverage container 108, the profiler 118 (refer to FIGS. 6A-7) and/or profiling arm 121 (refer to FIG. 9) can adjust to conform to the shape of the identified reusable beverage container 108, placing the one or more UV light source 120 in close proximity to the surface(s) of the reusable beverage container 108.

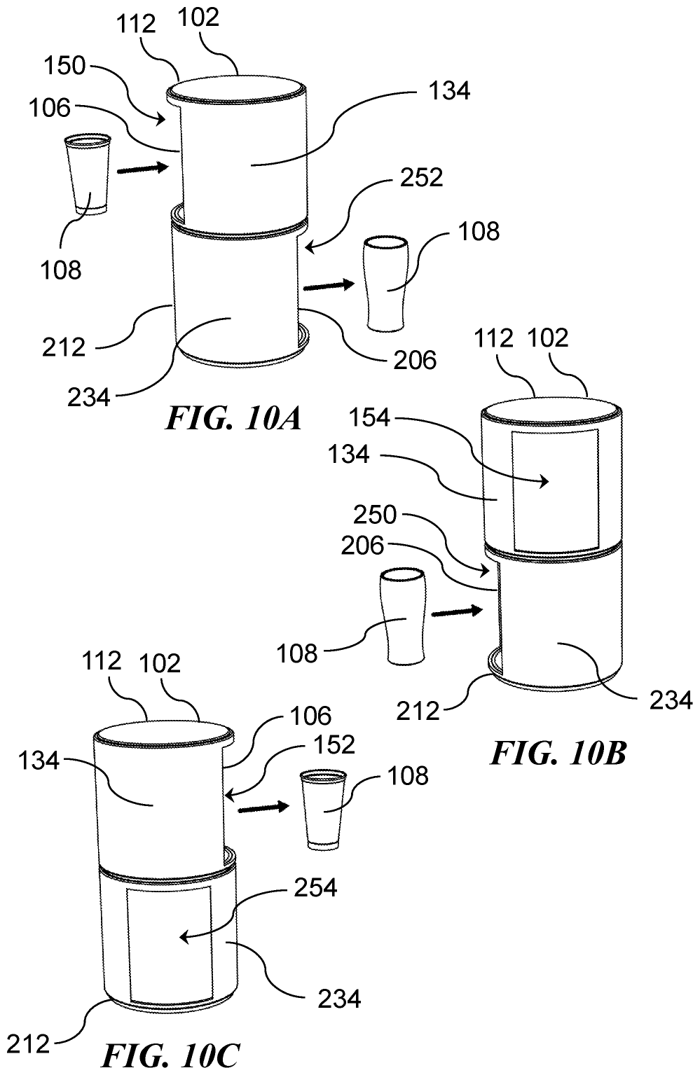

Even with the rapid sanitization cycle possible using the UV light source 120, the time required to achieve the appropriate level of sanitization may be too long when high throughput, or no waiting time to load, is desired. FIGS. 10A-10C show a dual stacked version of the revolving door system from FIG. 8. Here a second UV sanitizing subsystem 212 is provided that is sized, dimensioned, and configured to envelop a reusable beverage container 108. A second access door 206 is provided on second sliding member 234. The second access door 206 provides access to the second UV sanitizing subsystem 212 when the second access door 206 is in an open state and blocks access to the second UV sanitizing subsystem 212 when the second access door 206 is in a closed state. At least one UV light source 120 (not shown in this figure) is disposed within the second UV sanitizing subsystem 212 and configured to direct UV light at a predetermined intensity level and time duration onto a reusable beverage container 108 positioned inside the second UV sanitizing subsystem 212 in such a way as to result in sanitizing of surfaces of the reusable beverage container 108. The housing 102, in this case, the first sliding member 134 and second sliding member 234 of the housing 102, rotate to expose the “unsanitized” side opening, the “sanitized” side opening, or a closed position with no openings for the sanitizing process to take place within the UV sanitizing subsystem 112, 212. In FIG. 10A the first sliding member 134 of the housing 102 is in a first position 150 where the first access door 106 is in an open state for receiving an unsanitized reusable beverage container 108. The second sliding member 234 is in a second position 252 where the second access door 206 is in an open state allowing for the removal of a sanitized reusable beverage container 108 from the second UV sanitizing subsystem 212. In FIG. 10B the first sliding member 134 is moved such that the first access door 106 is in a closed state 154 during which a UV sanitization cycle is run. The second sliding member 234 is moved to a first position 250 where the second access door 206 is in an open state for receiving an unsanitized reusable beverage container 108. In FIG. 10C the first sliding member 134 is moved to a second position 152 where the first access door 106 is in an open state allowing for the removal of the now sanitized reusable beverage container 108. The second sliding member 234 has been moved such that the second access door 206 is in a closed state 254 during which a UV sanitization cycle is run in the second UV sanitizing subsystem 212. The sliding members 134, 234 can then be moved back to the positions shown in FIG. 10A.

By having dual UV sanitizing subsystems, such as the stacked subsystems as shown in FIGS. 10A-10C, or some other combination of two or more UV sanitizing subsystems, the overall through-put of the system is increased and wait times to use a system are decreased as one UV sanitizing subsystem can be loaded or unloaded while the other is running the UV sanitization cycle. These systems can be a single housing containing multiple UV sanitizing subsystems as shown in FIGS. 10A-10C, or multiple individual stand-alone UV sanitizing subsystems.

In certain embodiments, one of the first UV sanitizing subsystem 112 or second UV sanitizing subsystem 212 may be arranged or otherwise configured such that it operates in the opposite direction from the other UV sanitizing subsystem. For example, the first position 150 of the sliding member 134 may provide access to the “sanitized” side of the first UV sanitizing subsystems 112 where a sanitized reusable beverage container 108 can be retrieved and the second position 152 of the sliding member 134 provides access to the “unsanitized” side of the first UV sanitizing subsystems 112 where an unsanitized container can be inserted to be sanitized. Alternately, the second position 252 of the second sliding member 234 may be on the “unsanitized” side of the second UV sanitizing subsystems 212 and the first position 250 of the second sliding member 234 may be on the “sanitized” side of the second UV sanitizing subsystems 212. Such configurations allow bi-directional sanitization where a reusable beverage container 108 can be sanitized during both the transition of the reusable beverage container 108 from the customer to retail employee and from the retail employee to the customer.

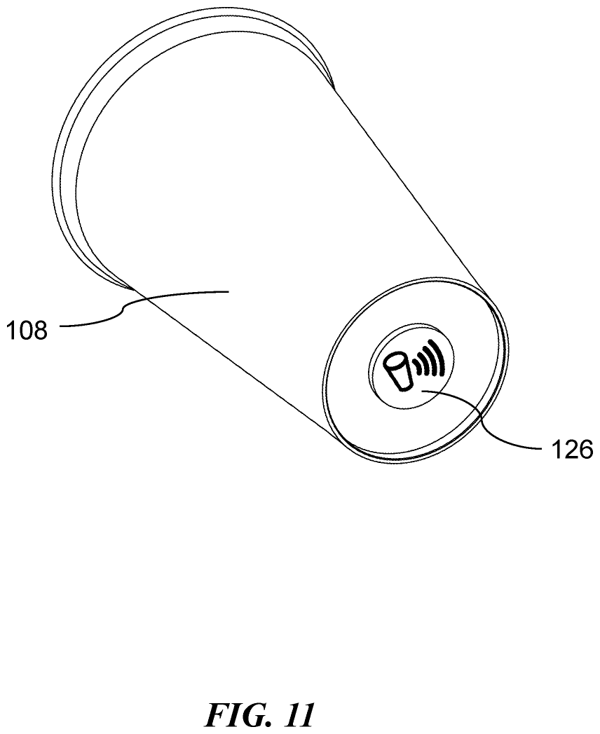

FIG. 11 shows an embodiment of the reusable beverage container 108 configured for use with the system 100 (refer to FIG. 1). The identification device 126 is incorporated in the reusable beverage container 108, such as an RFID tag, on or embedded within the reusable beverage container 108, and is read by devices of the system 100 (refer to FIG. 1), such as a reader 128 disposed at the UV sanitizing subsystem 112 (refer to FIG. 2), to identify when the reusable beverage container 108 is handed off from the customer to the retail establishment. Various different types of identification technologies, including but not limited to near-field communication (NFC) chip, bar code, quick response (QR) code, optical character recognition, or the like, are also feasible and can be implemented as the identification device 126 and corresponding reader 128 (refer to FIG. 1), as previously described.

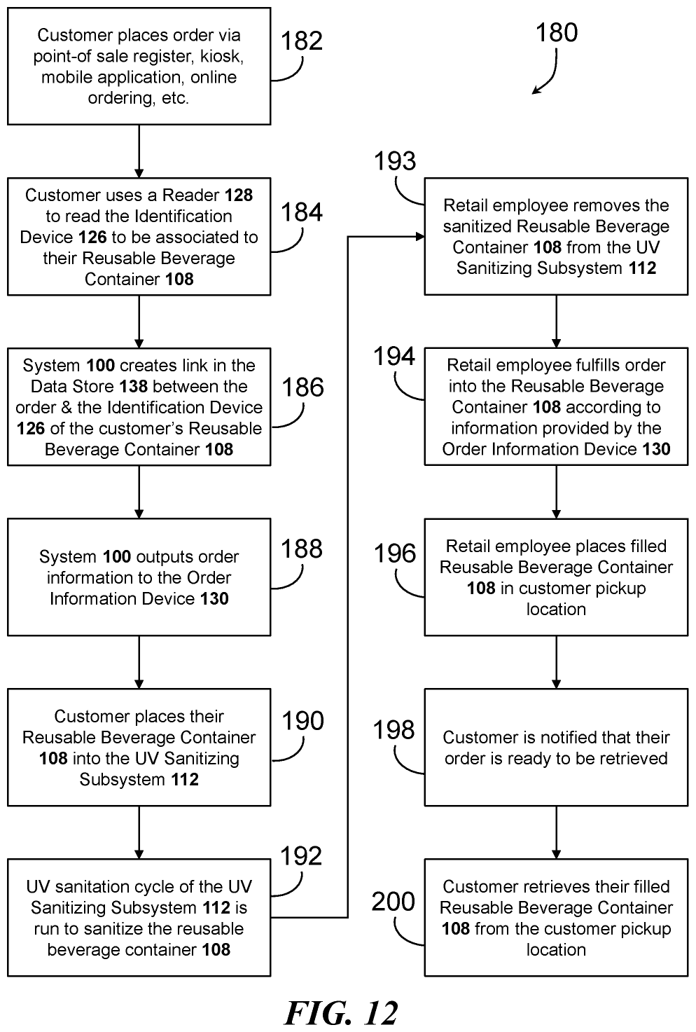

FIG. 12 is a flow diagram of an example embodiment of the reusable beverage container sanitizing system process 180 in operation that associates a unique identification device 126 of a reusable beverage container 108 to a customer order such that the system 100 associates a specific order to be fulfilled in that reusable beverage container 108 after it has been sanitized using the UV sanitizing subsystem 112. Specifically, the customer orders a beverage either directly at a point-of-sale register, kiosk, using a mobile smartphone application, or online ordering, and the like (step 182), then uses a reader 128 of the identification subsystem 160 to read the identification device 126 to be associated to their reusable beverage container 108 (step 184). The system 100 creates an association in the data store 138 between the order and that identification device 126 associated with that reusable beverage container 108 (step 186) and outputs that information to the order information device 130 (step 188). The customer places their reusable beverage container 108 into the UV sanitizing subsystem 112 (step 190) and the UV sanitization cycle is run (step 192). Once the UV sanitization cycle is complete, the retail employee removes the reusable beverage container 108 from the UV sanitizing subsystem 112 (step 193) and fulfills the order in that reusable beverage container 108 according to the information provided by the order information device 130 (step 194). Once the order has been fulfilled, the filled reusable beverage container is placed at the customer pickup location (step 196), the customer is notified that their order is ready to be retrieved (step 198) and the customer retrieves their filled reusable beverage container 108 from the customer pickup location (step 200). As previously described and appreciated by one of skill in the art, the specific sequence of operations, and location and usage of devices may be altered while still keeping with the spirit and scope of the invention.

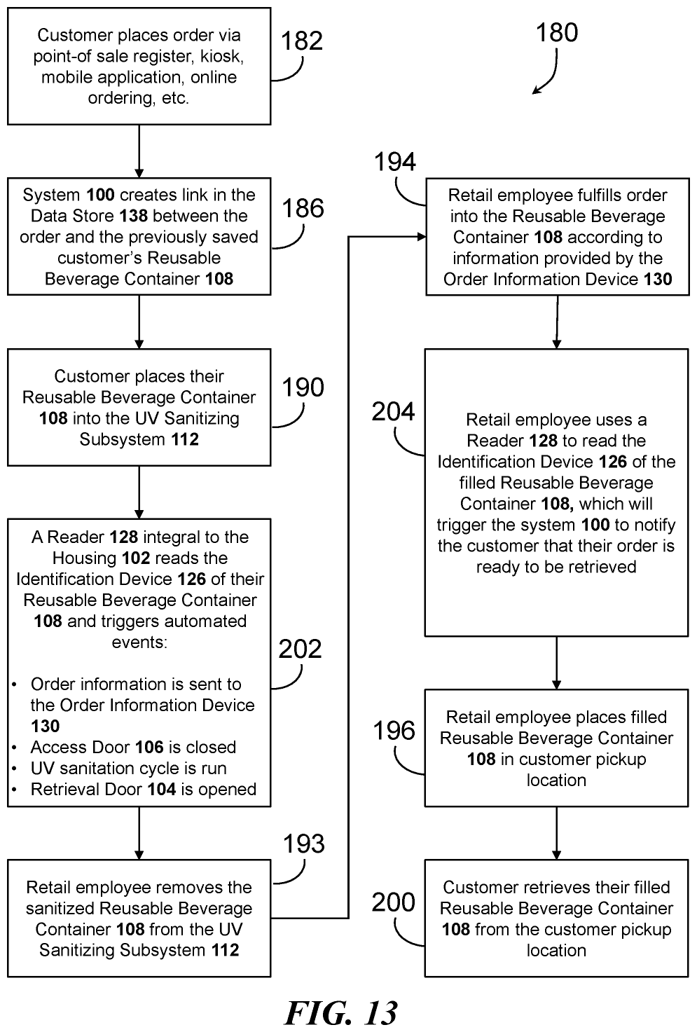

FIG. 13 is a flow diagram of an example embodiment of the reusable beverage container sanitizing system process 180 using automated processes of the system 100 to enhance the speed and ease of use of the system 100 for both the customer and the retail employee. Once the customer has previously used the system 100, the information pertaining to the identification device 126 on or embedded within the customer's reusable beverage container 108 is saved in the data store 138. As such, when the known customer orders a beverage (step 182), the link between the customer's order and the reusable beverage container 108 will be created automatically as soon as the customer places the order (step 186), as previously described. Alternatively, the customer can save a specific order for that reusable beverage container 108 such that the order will be placed automatically when the identification device 126 of the reusable beverage container 108 is read by a reader 128 of the identification subsystem 160.

With the link established between the customer's order and the reusable food or beverage container 108, the customer places their reusable food or beverage container 108 into the UV sanitizing subsystem 112 (step 190). A reader 128 integral to the housing 102 of the UV sanitizing subsystem 112 reads the identification device 126 of the reusable beverage container 108. In the exemplary embodiment, the successful reading of identification device 126 triggers a series of automated events (step 202): order information is sent to the order information device 130, the access door 106 is closed, the UV sanitization cycle is run, and the retrieval door 104 is open once the UV sanitization cycle is complete. The retail employee then removes the reusable beverage container 108 from the UV sanitizing subsystem 112 (step 193) and fulfills the order in that reusable beverage container 108 according to the information provided by the order information device 130 (step 194). Once the order has been fulfilled, the identification device 126 of the filled reusable beverage container 108 is read by a reader 128 (step 204) which will trigger the system 100 to automatically notify the customer, via text message, mobile application notification, audio or video alert within the retail establishment, and the like, that their order is ready to be retrieved. The retail employee then places the filled reusable beverage container 108 in the customer pick-up area (step 196) where it is retrieved by the customer (step 200). This exemplary embodiment demonstrates one configuration of devices of the system for enhancing automation. As previously described and appreciated by one of skill in the art, the specific sequence of operations, and location and usage of devices may be altered while still keeping with the spirit and scope of the invention.

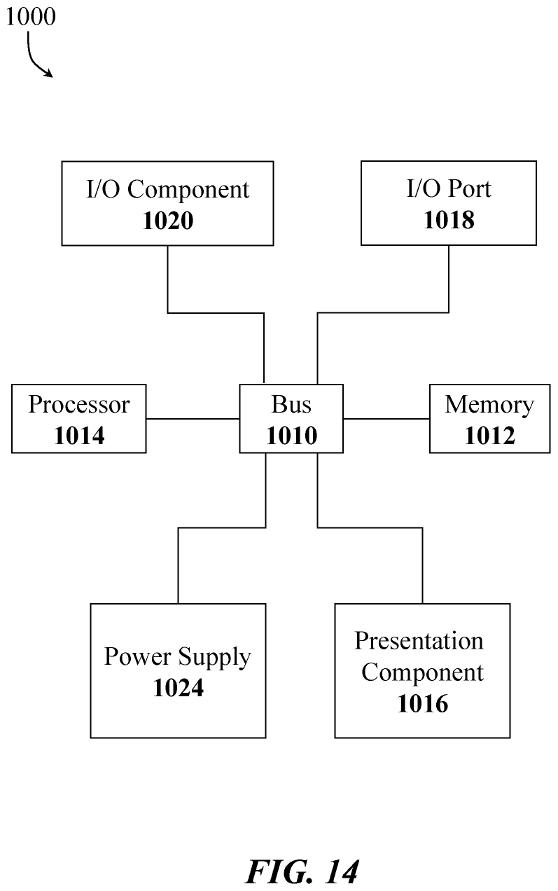

FIG. 14 depicts an example electronic, computer, or computing device 1000 that can be used to implement one or more aspects of the present invention. The functionality and hardware of such computing device 1000 may be implemented in any of the electronic hardware systems or subsystems described herein as involving or using a “computer” or “computing device” or the like, or related hardware for providing all or part of the described functionality, provided as a separate device or integrated into a system or subsystem described herein, as would be appreciated and understood by those of skill in the art. The terms “computer”, “computing device”, and the like utilized herein are intended to mean a processor at its most basic form, on up to more complex computing systems, including servers and cloud-based systems, in accordance with conventional meanings of such terms. However, for purpose of completeness, example components and related accessories that are intended to be encompassed by the use of the terms “computer”, “computing device”, “processor”, and the like will be provided below in example nonlimiting form.

The computing device 1000 is merely an illustrative example of a suitable computing environment and in no way limits the scope of the present invention. An “electronic device”, “remote device,” or “personal electronic device” as represented in figures and description herein, can include a “workstation,” a “server,” a “laptop,” a “desktop,” a “hand-held device,” a “mobile device,” a “tablet computer,” a “processor,” or other computing devices, as would be understood by those of skill in the art. Given that the computing device 1000 is depicted for illustrative purposes, embodiments of the present invention may utilize any number of computing devices 1000 in any number of different ways to implement a single embodiment of the present invention. Accordingly, embodiments of the present invention are not limited to a single computing device 1000, as would be appreciated by one with skill in the art, nor are they limited to a single type of implementation or configuration of the example computing device 1000.

The computing device 1000 can include a bus 1010 that can be coupled to one or more of the following illustrative components, directly or indirectly: a memory 1012, one or more processors 1014, one or more presentation components 1016, input/output ports 1018, input/output components 1020, and a power supply 1024. One of skill in the art will appreciate that the bus 1010 can include one or more busses, such as an address bus, a data bus, or any combination thereof. One of skill in the art additionally will appreciate that, depending on the intended applications and uses of a particular embodiment, multiple of these components can be implemented by a single device. Similarly, in some instances, a single component can be implemented by multiple devices. As such, the figures herein are merely illustrative of an exemplary computing device 1000 that can be used to implement one or more embodiments of the present invention, and in no way limits the invention.

The computing device 1000 can include or interact with a variety of computer-readable media. For example, computer-readable media can include Random Access Memory (RAM); Read Only Memory (ROM); Electronically Erasable Programmable Read Only Memory (EEPROM); flash memory or other memory technologies; CDROM, digital versatile disks (DVD) or other optical or holographic media; magnetic cassettes, magnetic tape, magnetic disk storage or other magnetic storage devices that can be used to encode information and can be accessed by the computing device 1000.

The memory 1012 can include computer-storage media in the form of volatile and/or nonvolatile memory. The memory 1012 may be removable, non-removable, or any combination thereof. Exemplary hardware devices are devices such as hard drives, solid-state memory, optical-disc drives, and the like. The computing device 1000 can include one or more processors that read data from components such as the memory 1012, the various I/O components 1020, etc. Presentation component(s) 1016 present data indications to a user or other device. Exemplary presentation components include a display device, speaker, printing component, vibrating component, etc.

The I/O ports 1018 can enable the computing or electronic device 1000 to be logically coupled to other devices, such as I/O components 1020. Some of the I/O components 1020 can be built into the computing device 1000. Examples of such I/O components 1020 include a sensor (including but not limited to: weight sensor, infrared sensor, camera, chemical sensor, microphone, or the like), keypad, touchpad, joystick, recording or storage device, game pad, satellite dish, scanner, printer, wireless device, networking device, and the like, as appropriate.

To any extent utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about” and “approximately” are intended to cover variations that may exist in the upper and lower limits of the ranges of subjective or objective values, such as variations in properties, parameters, sizes, and dimensions. In one non-limiting example, the terms “about” and “approximately” mean at, or plus 10 percent or less, or minus 10 percent or less. In one non-limiting example, the terms “about” and “approximately” mean sufficiently close to be deemed by one of skill in the art in the relevant field to be included. As utilized herein, the term “substantially” refers to the complete or nearly complete extent or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present invention will be apparent to those skilled in the art in view of the foregoing description. Accordingly, this description is to be construed as illustrative only and is for the purpose of teaching those skilled in the art the best mode for carrying out the present invention. Details of the structure may vary substantially without departing from the spirit of the present invention, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present invention be limited only to the extent required by the appended claims and the applicable rules of law.

It is also to be understood that the following claims are to cover all generic and specific features of the invention described herein, and all statements of the scope of the invention which, as a matter of language, might be said to fall therebetween.

What is claimed is:

1. A reusable food or beverage container sanitizing system, comprising:
a reusable food or beverage container;
an identification subsystem, comprising:
an identification device associated with the reusable food or beverage container;
at least one reader that communicates with the identification device and exchanges container data that characterizes the identification device and associated reusable food or beverage container; and
a computing device in communication with a data store storing information relating a food or beverage order to be fulfilled in the reusable food or beverage container;
an order information device; and
a UV sanitizing subsystem comprising:
a housing sized, dimensioned, and configured to envelop a reusable food or beverage container; and
at least one UV light source configured to direct UV light at a predetermined intensity level and time duration onto a reusable food or beverage container in such a way as to result in sanitizing of surfaces of the reusable food or beverage container;
wherein the UV sanitizing subsystem receives an unsanitized reusable food or beverage container;
wherein the UV sanitizing subsystem sanitizes the reusable food or beverage container prior to the food or beverage order being fulfilled in the reusable food or beverage container;
wherein the food or beverage order is fulfilled in the sanitized reusable food or beverage container according to information provided by the order information device in communication with the data store.

2. The reusable food or beverage container sanitizing system of claim 1, wherein an instance of the at least one reader is disposed to read the identification device prior to placing the reusable food or beverage container into the UV sanitizing subsystem.

3. The reusable food or beverage container sanitizing system of claim 1, wherein the identification device is incorporated on or embedded within the reusable beverage container.

4. The reusable food or beverage container sanitizing system of claim 3, wherein an instance of the at least one reader is disposed to read the identification device of the reusable beverage container when placed into the UV sanitizing subsystem.

5. The reusable food or beverage container sanitizing system of claim 3, wherein an instance of the at least one reader is disposed to read the identification device of the reusable beverage container prior to returning a filled container to a customer.

6. The reusable food or beverage container sanitizing system of claim 1, wherein the identification device comprises one or more of a bar code, a quick response (QR) code, a near-field communication (NFC) chip, or a radio-frequency identification (RFID) tag.

7. The reusable food or beverage container sanitizing system of claim 1, wherein the order information device is a receipt or label printer.

8. The reusable food or beverage container sanitizing system of claim 1, wherein the order information device is a display screen.

9. The reusable food or beverage container sanitizing system of claim 1, wherein housing of the UV sanitizing subsystem comprises an access door providing access to the UV sanitizing subsystem when the access door is in an open state and blocking access to the UV sanitizing subsystem when the access door is in a closed state.

10. The reusable food or beverage container sanitizing system of claim 9, wherein when the access door is in the closed state, a UV sanitization cycle is initiated using the UV light source and when the UV sanitization cycle is complete, the access door is moved to an open state.

11. The reusable food or beverage container sanitizing system of claim 10, wherein the UV sanitizing subsystem further comprises a retrieval door opposite the access door, the retrieval door providing access to the UV sanitizing subsystem when the retrieval door is in an open state and blocking access to the UV sanitizing subsystem when the retrieval door is in a closed state.

12. The reusable food or beverage container sanitizing system of claim 11, wherein the access door is configured to receive an unsanitized reusable food or beverage container and the retrieval door is configured to allow retrieval of a sanitized reusable food or beverage container after the UV sanitization cycle.

13. The reusable food or beverage container sanitizing system of claim 12, further including a conveyor configured to receive an unsanitized reusable food or beverage container, move the unsanitized reusable food or beverage container in the UV sanitizing subsystem through the access door, and move the sanitized reusable food or beverage container out of the UV sanitizing subsystem through the retrieval door.

14. The reusable food or beverage container sanitizing system of claim 13, wherein the conveyor comprises a conveyor belt.

15. The reusable food or beverage container sanitizing system of claim 9, further comprising a container centering device disposed within the UV sanitizing subsystem.

16. The reusable food or beverage container sanitizing system of claim 15, wherein the container centering device comprises at least one mechanical arm.

17. The reusable food or beverage container sanitizing system of claim 9, wherein the at least one UV light source comprises an array of UV lights.

18. The reusable food or beverage container sanitizing system of claim 17, wherein the array of UV light is disposed on an inner surface of one or more sides of the UV sanitizing subsystem.

19. The reusable food or beverage container sanitizing system of claim 9, wherein the at least one UV light source is disposed on a container profiler that is configured to conform to a shape of the reusable food or beverage container to place the at least one UV light source in proximity to an outer surface of the reusable food or beverage container allowing for effective sanitization at a lower intensity level or time duration.

20. The reusable food or beverage container sanitizing system of claim 19, wherein the profiler comprises one or more members having at least one UV light source mounted thereon, the members configured to move toward an outer surface of the reusable food or beverage container to place the UV light in proximity to the outer surface of the reusable food or beverage container.

21. The reusable food or beverage container sanitizing system of claim 20, wherein the container profiler comprises a plurality of linear moving members.

22. The reusable food or beverage container sanitizing system of claim 20, wherein the container profiler comprises articulating members.

23. The reusable food or beverage container sanitizing system of claim 19, wherein the profiler comprises one or more sensors for detecting a shape of the reusable food or beverage container.

24. The reusable food or beverage container sanitizing system of claim 19, wherein the container profiler is configured to rotate around the container to distribute application of UV light.

25. The reusable food or beverage container sanitizing system of claim 9, further comprising a rotating base for rotating the reusable food or beverage container within the UV sanitizing subsystem.

26. The reusable food or beverage container sanitizing system of claim 9, wherein the at least one UV light source is disposed on an arm that extends into an internal volume of the reusable food or beverage container to sanitize an interior surface of the container.

27. The reusable food or beverage container sanitizing system of claim 26, wherein the arm is configured to place the UV light source in proximity to the interior surface of the reusable food or beverage container allowing for effective sanitization at a lower intensity level or time duration.

28. The reusable food or beverage container sanitizing system of claim 26, wherein the arm is configured to rotate within the internal volume of the container to distribute application of UV light.

29. The reusable food or beverage container sanitizing system of claim 9, wherein the housing rotates around the UV sanitizing subsystem and wherein the access door connected to the housing receives unsanitized reusable food or beverage container in a first position located on a first side of the UV sanitizing subsystem and rotates to a second position located on second side of the UV sanitizing subsystem to provide access to a sanitized reusable food or beverage container.

30. The reusable food or beverage container sanitizing system of claim 9, wherein sliding members of the housing rotate around the UV sanitizing subsystem and wherein the sliding members provide an access door which receives unsanitized reusable food or beverage container in a first position located on a first side of the UV sanitizing subsystem and rotate to a second position located on second side of the UV sanitizing subsystem to provide access to a sanitized reusable food or beverage container.

31. The reusable food or beverage container sanitizing system of claim 9, wherein the housing includes a cleansing device.

32. The reusable food or beverage container sanitizing system of claim 9, further comprising:
a second UV sanitizing subsystem inside the housing that is sized, dimensioned, and configured to envelop a reusable food or beverage container;
a second access door providing access to the second UV sanitizing subsystem when the second access door is in an open state, and blocking access to the second UV sanitizing subsystem when the second access door is in a closed state; and
at least one UV light source disposed within the second UV sanitizing subsystem and configured to direct UV light at a predetermined intensity level and time duration onto the reusable food or beverage container positioned inside the second UV sanitizing subsystem in such a way as to result in sanitizing of surfaces of the reusable food or beverage container;
wherein when the second access door is in the closed state a UV sanitization cycle is initiated using the UV light source and when the UV sanitization cycle is complete, the second access door is moved to the open state.

33. The reusable food or beverage container sanitizing system of claim 32, wherein the second UV sanitizing subsystem is configured to operate in an opposite direction of the UV sanitizing subsystem.

34. The reusable food or beverage container sanitizing system of claim 9, wherein the access door is locked during a sanitization cycle and unlocked after the sanitization cycle.

35. The reusable food or beverage container sanitizing system of claim 9, wherein the access door automatically transitions between the closed and open states.

36. A method of enabling use of reusable food or beverage containers using a reusable food or beverage container sanitizing system, the method comprising:
providing a reusable food or beverage container sanitizing system comprising a reusable food or beverage container, an identification subsystem with at least one reader, an order information device, and a UV sanitizing subsystem, the reusable food or beverage container having an associated identification device readable by the at least one reader;
receiving a customer order;
reading the identification device associated to the customer's reusable food or beverage container;
associating the customer order to the reusable food or beverage container;
the customer placing the reusable food or beverage container into the UV sanitizing subsystem;
the UV sanitizing subsystem sanitizing the reusable food or beverage container;
a retail employee retrieving the sanitized reusable food or beverage container from the UV sanitizing subsystem;
the retail employee fulfilling the customer order in the reusable food or beverage container according to information provided by the order information device; and
customer retrieving the fulfilled order in the reusable food or beverage container from a customer pickup location.
